(19) United States
(12) Patent Application Publication
(10) Pub. No.: US 2011/0046557 A1
(43) Pub. Date: Feb. 24, 2011

(54) MICRONEEDLE DRUG DELIVERY SYSTEM INCLUDING MOVABLE DRUG-CONTAINING CAPSULE

(75) Inventors: Seung Seob Lee, Daejeon (KR); Boo Joon Sul, Daejeon (KR); Man Hee Han, Daejeon (KR)

Correspondence Address: Edwards Angell Palmer & Dodge LLP, P.O. Box 55874, Boston, MA, 02205, US

(73) Assignee: MITI SYSTEMS INC., Daejeon (KR)

(21) Appl. No.: 12/894,924

(22) Filed: Sep. 30, 2010

Related U.S. Application Data
(63) Continuation of application No. PCT/KR2010/003022, filed on May 13, 2010.

(30) Foreign Application Priority Data
Aug. 4, 2009 (KR) 10-2009-0071572

(51) Int. Cl. A61M 5/00

(52) U.S. Cl. 604/173

(57) ABSTRACT

A microneedle drug delivery system comprises: a housing having an opening formed in the bottom wall thereof; a microneedle device seated in the opening of the housing in such a fashion as to be hermetically sealed with the bottom wall of the housing; a drug-containing capsule mounted in the housing 1 in such a fashion as to be positioned spaced apart from the microneedle device. With this system, an active agent (or drug) can be effectively delivered into a subject in need.

### CROSS-REFERENCE TO RELATED APPLICATION

This is a continuation of International Application No. PCT/KR2010/003022, with an international filing date of May 13, 2010, which claims the benefit of Korean Application No. 10-2009-0071572 filed Aug. 4, 2009, the entire contents of which are incorporated herein by reference.

### TECHNICAL FIELD

The present invention relates to a microneedle drug delivery system that does not cause the leakage of an active agent therein to the outside even in the event of an external impact or shock occurred during distribution and storage of the system, can be carried and used more simply and conveniently, and can effectively deliver an active agent to a subject in need.

### BACKGROUND ART

The oldest method for delivering a drug into the human body is a method which orally administers a drug. But, such an oral administration method involves a problem in that the concentration of the drug is difficult to constantly maintain in the body apart from aversion to the drug upon the administration of the drug, and the drug administered into the body is dissolved by digestive organs or is filtered by a liver. In the meantime, a method of percutaneously administering a drug in which a drug is applied on the skin or a pack, a patch or the like containing a drug is attached to the skin has a merit in that the drug can be very simply and easily brought into close contact with the skin and the concentration of the drug can be constantly maintained by using the patch. However, such a transdermal administration method still has a shortcoming in that since the stratum corneum which is an outermost layer of the epidermis of the skin and is 10-60 μm in depth inhibits the penetration of external substances into the human body, transdermal absorption of the drug is extremely low. In particular, if the drug is hydrophilic or has a high molecule weight, the transdermal absorption of the drug further decreases.

As a solution to this problem, a method has been used which injects or administers a drug into the human body through a syringe in order to effectively transfer the drug into the body. A conventional syringe needle has a diameter measured in millimeter units (mm) and a length measured in centimeter units (cm). Such a syringe needle stimulates a plurality of pain spots widely distributed in the skin, which gives a considerable pain to a subject in use. In addition, this intracutaneous injection method using the syringe entails a drawback in that since it is mainly used in a hospital or a professional skin care agency, it cannot be readily utilized in general homes.

In order to address and solve the above drawback, a microneedle has been developed which has a diameter of several tens to a few hundreds of micrometers (μm) and a length of several tens to a few thousands of micrometers (μm). Since this microneedle is relatively small in diameter and length as compared to the conventional syringe needles, the number of pain spots stimulated is reduced, thereby resulting in significant alleviation of a pain given to the subject and making its use in general homes convenient. Besides, the microneedle is excellent in drug in vivo permeability or drug delivery durability. Therefore, it is expected that the microneedle will be substituted as a new drug delivery system.

In order to eliminate an inconvenience of having to separately be provided with microneedles and a drug, as an example of a conventional microneedle drug delivery system, U.S. Pat. No. 3,964,482 discloses an integral-type drug delivery device which includes a plurality of needle-like puncturing projections and a drug reservoir in immediate proximity with the needle-like projections, each having a fluid passageway or channel formed therein so as to allow a drug from the reservoir to enter the projections and then to be percutaneously transferred into the body therethrough. However, the above U.S. patent has a disadvantage in that since there is no membrane or diaphragm formed between the drug reservoir and the channel through which the drug passes, the drug contained in the drug reservoir may leak to the outside during distribution and storage. Thus, it is impossible to apply the integral-type microneedle drug delivery system to not only a liquid-phase drug but also a gel-type drug.

In order to solve such a problem, there has been proposed an example of a conventional microneedle drug delivery system as shown in FIG. 1. That is, U.S. Pat. No. 6,656,147 discloses a delivery device for the transdermal administration of a substance in which a drug reservoir, i.e., a prefilled bladder containing therein a drug is positioned between a microneedle substrate, i.e., a planar member integrally formed with microneedles and a cover, and a cannula is provided on the top surface of the planar member so as to pierce the bladder. When pressure is applied to the cover, the cannula pierces and collapses the bladder to cause the drug contained in the bladder to be transferred to tips of the microneedles, i.e., the skin surface through passages or channels formed in the planar member. In addition, as another example of the conventional microneedle drug delivery system, U.S. Patent Application Publication No. 2009/0030365 discloses a transdermal drug administration apparatus having microneedles, which can apply a drug that should be kept in a solid state upon distribution and custody due to unstability of the drug in a solution state. Such a transdermal drug administration apparatus of the U.S. patent includes an absorbent disposed on a microneedle substrate so as to store a solid-phase drug therein and absorb a solvent or a dissolving solution for dissolving the drug, a solvent reservoir disposed on the absorbent and containing the solvent, so that when the reservoir is pressed, a diaphragm provided between the reservoir and the absorbent is disrupted to cause the drug dissolved in the solvent to be delivered into the skin through the microneedles. However, the above U.S. patent has a demerit in that when an impact is externally exerted on the apparatus in the course of distribution and storage, the diaphragm or membrane is broken or disrupted to cause the drug dissolved in the dissolving solution to be lost.

As yet another example of the conventional microneedle drug delivery system, U.S. Patent Application Publication No. 2007/0021717 discloses a transdermal delivery device which includes: a disposable cartridge having micro skin penetrating members serving as microneedles, an internal reservoir containing a drug to be delivered to the skin, and a piercing assembly for piercing a seal serving as a side wall of the reservoir; and a housing adapted to receive the cartridge therein. According to the above transdermal delivery device, the cartridge mounted inside the housing is pressed downwardly so that the drug contained in the reservoir is transferred to the microneedles. However, such a transdermal delivery device entails a shortcoming in that it is separately provided with the cartridge and the housing, and the entire system is complicated, leading to an increase in the product cost. In addition, the transdermal delivery device involves a problem in that if the housing is not hygienically managed due to the repeated use of the housing, the risk of bacterial infection increases at the time of application of the microneedles.

As such, the above drug delivery systems using the microneedles are greatly high in the drug compliance of a patient and effectively in clinical practice. Nevertheless, there still is a need to develop a microneedle drug delivery system which can be more conveniently and sanitarily while facilitating its storage and distribution.

The above information disclosed in this Background section is only for enhancement of understanding of the background of the invention and therefore it may contain information that does not form the prior art that is already known in this country to a person of ordinary skill in the art.

### SUMMARY OF THE DISCLOSURE

The present invention has been made in order to solve the above-described problems occurring in the prior art, and it is an object of the present invention to provide a microneedle drug delivery system in which a drug and a microneedle device for drug delivery are integrally formed with each other so that the use of the system is convenient, and a liquid-phase drug as well as a solid or gel-type drug can be easily carried and can be effectively delivered in vivo through the skin with no or less pain.

Another object of the present invention is to provide a microneedle drug delivery system which can be securely protected from an external impact or shock occurred during distribution and storage thereof to prevent a drug from leaking to the outside and can be safely used without any risk of the contamination.

To achieve the above objects, the present invention provides a microneedle drug delivery system including: a housing having an opening formed in the bottom wall thereof; a microneedle device including a substrate, a microneedle array formed protrudingly downwardly from the bottom surface of the substrate so as to be able to pierce the skin, one or more capsule-disrupting micro-projections formed upwardly from the top surface of the substrate, and one or more drug delivery channels formed therein so as to allow a drug to be delivered from the top surface of the substrate to the bottom surface of the substrate therethrough, wherein the microneedle device is seated in the opening of the housing in such a fashion as to be hermetically sealed with the bottom wall of the housing; and a drug-containing capsule mounted in the housing in such a fashion as to be positioned spaced apart from the microneedle device, and adapted to be moved to a position where the drug-containing capsule can come into contact with the one or more capsule-disrupting micro-projections to allow the drug-containing capsule to be disrupted by the one or more capsule-disrupting micro-projections.

According to the microneedle drug delivery system of the present invention, since the drug-containing capsule is positioned spaced apart from the microneedle device, the drug-containing capsule is prevented from being disrupted or perforated by the contact with the capsule-disrupting micro-projections due to an external impact, which can occur upon the storage and distribution, to cause the drug in the capsule to be lost. During the use of the microneedle drug delivery system, the drug-containing capsule is moved to a position where it can be brought into contact with the capsule-disrupting micro-projections. Then, when the drug-containing capsule is pressed with a certain pressure, it is disrupted or broken by the capsule-disrupting micro-projections so that the drug flowing out of the capsule is delivered into the skin through the drug delivery channels and simultaneously is the microneedles penetrate the skin to allow the drug to be delivered in vivo. The drug-containing capsule and the microneedle device may be constructed such that they are horizontally or vertically spaced apart from each other.

Preferably, the region in which the microneedle device is seated defines a concave-shaped drug-accommodating portion at the bottom wall of the housing so that the drug flowing out of the capsule upon disruption of the capsule can be prevented from being diffused to other places to cause the drug to be lost and the drug can be efficiently delivered to the skin.

Formation of the drug delivery channels in the capsule-disrupting micro-projections or the microneedles is relatively difficult in a technical aspect as compared to formation of the drug delivery channels in the substrate, thereby contributing to an increase in the manufacturing cost. On the other hand, in the case where the drug delivery channels are penetratingly formed in the microneedles, the drug is directly delivered in vivo through the microneedles so that it can be more effectively administered in an accurate dosage. On the contrary, in the case where the drug delivery channels are formed in the substrate, the drug may be not introduced in vivo but may flow along the outer surface of the skin. Thus, the drug delivery channels are formed in a different manner depending on the use and purpose of the microneedle drug delivery system. That is, in the case where the drug delivery efficiency or the drug delivery amount is not significantly important, the drug delivery channels may preferably be formed penetratingly in the substrate, and in the case where the drug delivery efficiency or the drug delivery amount is important despite an increase in the manufacturing cost, the drug delivery channels may preferably be formed penetratingly in the substrate and microneedles.

Preferably, the housing may further include a fixing cap disposed on the top thereof so as to prevent displacement of the drug-containing capsule therein at normal times and so as to be removable therefrom in use. The fixing cap may take all types of structures as long as it can serve to prevent displacement of the drug-containing capsule. That is, as shown in FIGS. 3 and 4, the fixing cap may be formed in a plate shape which covers the top of the capsule-disrupting micro-projections, and may be formed in the shape of a protruding pin which merely blocks the movement path of the drug-containing capsule. In addition, although a concrete shape of the fixing cap is not described separately, those skilled in the art will adopt a fixing cap having a variety of structures which can prevent displacement of the drug-containing capsule and can be easily removed in use through conventional prior arts.

Moreover, the housing may further include a cover disposed on the top thereof so as to protect the top of the drug-containing capsule. The cover functions to prevent the drug-containing capsule from escaping from the housing as well as further protect the drug-containing capsule from an external impact.

Also, the microneedle array may further include a microneedle tip protection cover such as a film or lid provided at the bottom thereof so as to prevent damage and contamination of a tip of the microneedle.

The drug-containing capsule may be divided into two internal compartments so as to store more than two drug ingredients. In the complex prescription case of taking a combination drug containing more than two ingredients, if the drug exists in a mixed state, its stability may be deteriorated. In this case, if the drug ingredients causing deterioration of stability upon the contact therebetween are separately stored in the divided internal compartments of the drug-containing capsule, they do not come into contact with each other so that the stability is maintained. Also, the capsule is disrupted by one-time operation and simultaneously the drug contained in the capsule can be delivered in vivo. When the capsule having divided internal compartments is used, a solid or gel-type drug unstable in the liquid phase as well as a liquid-phase drug can be delivered in vivo. In order to apply the solid or gel-type drug, the drug-containing capsule may be preferably divided into two internal compartments in a horizontal configuration or in a dual capsule configuration in which another capsule is included in a main capsule. For example, an upper compartment of the horizontally divided capsule or an inner compartment of the dual capsule may be filled with a solvent, and a lower compartment of the horizontally divided capsule or an outer compartment of the dual capsule may be filled with a solid or gel-type drug. Upon the administration of the drug, when the capsule is disrupted by the capsule-disrupting micro-projections, the solvent flows out of the upper or inner compartment of the capsule and dissolves the drug contained in the lower or outer compartment of the capsule so as to allow the dissolved drug to be delivered in vivo.

The microneedle drug delivery system according to the present invention may be used for delivery of a solid or gel-type drug by forming a solid or gel-type drug layer on the top surface of the capsule-disrupting micro-projections or on the bottom surface of the microneedles for piercing the skin, and filling a solvent capable of dissolving the drug in the drug-containing capsule. In this case, when the drug-containing capsule is disrupted by the capsule-disrupting micro-projections, the solvent flows out of the capsule and dissolves a solid or gel-type drug contained in the drug layer so that the dissolved drug can be delivered in vivo. At this time, in the case where the drug layer is formed on the bottom surface of the microneedles, a microneedle tip protection film for preventing separation of the drug layer and damage of the tips of the microneedles is preferably additionally provided on the bottom of the microneedle array. In order to protect the drug layer from a physical stimulus and extend the time during which the drug layer is brought into contact with the solvent contained in the drug-containing capsule, the drug layer is more preferably formed on the top surface of the capsule-disrupting micro-projections.

As described above, according to the present invention, it is possible to provide a microneedle drug delivery system in which the drug and the microneedle device are integrally formed with each other so that they can be carried together and stored integrally, and which can be stably maintained without any leakage of the drug to the outside due to an external impact during the storage of the system. Thus, a hydrophilic or high molecule weight drug making the transdermal absorption of the drug difficult can be delivered in vivo more conveniently.

Furthermore, since the microneedle drug delivery system of the present invention can be applied to the integral-type microneedle drug delivery system to not only a liquid-phase drug but also a solid or gel-type drug, it is possible to further expand the range in which the drug can be delivered transdermally without any pain.

The above and other features and advantages of the present invention will be apparent from or are set forth in more detail in the accompanying drawings, which are incorporated in and form a part of this specification, and the following Detailed Description, which together serve to explain by way of example the principles of the present invention.

### BRIEF DESCRIPTION OF THE DRAWINGS

FIG. 1 is a cross-sectional view illustrating an example of a conventional microneedle drug delivery system according to the prior art;

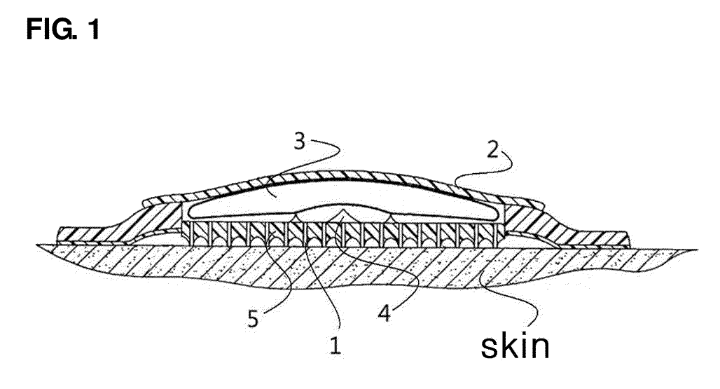

FIG. 2A to 2C are perspective and cross-sectional views illustrating a microneedle drug delivery system according to an embodiment of the present invention;

FIG. 3A to 3D are perspective and cross-sectional views illustrating the use example of a microneedle drug delivery system according to an embodiment of the present invention;

FIG. 4 is a perspective cross-sectional view illustrating a microneedle drug delivery system according to another embodiment of the present invention; and

FIG. 5A to 5D are cross-sectional views illustrating various examples of a microneedle device of a microneedle drug delivery system according to the present invention.

### DETAILED DESCRIPTION OF THE DISCLOSURE

Hereinafter, the present invention will be described in detail in connection with the preferred embodiments with reference to the accompanying drawings. However, these embodiments are for illustrative purposes, and the scope of the present invention is not limited thereto. Also, it will be understood by those skilled in the art that various modifications and variations can be made to the present invention without departing from the spirit and scope of the appended claims based on the illustrative embodiments.

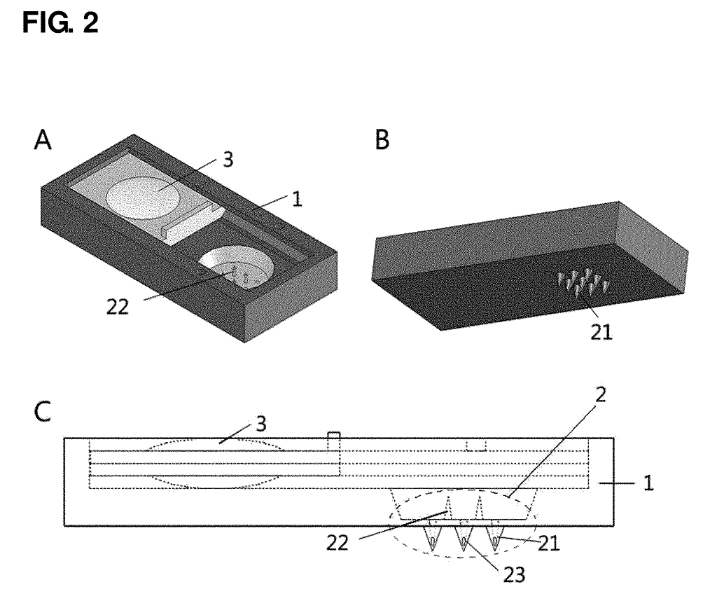

FIG. 2 illustrates a microneedle drug delivery system including a movable drug-containing capsule according to an embodiment of the present invention.

The microneedle drug delivery system according to the embodiment of the present invention includes a housing 1, a microneedle device 2, and a drug-containing capsule 3. The housing 1 has an opening formed in the bottom wall thereof. The microneedle device 2 is seated in the opening of the housing in such a fashion as to be hermetically sealed with the bottom wall of the housing 1. The drug-containing capsule 3 is mounted in the housing 1 in such a fashion as to be positioned spaced apart from the microneedle device 2 by a predetermined distance.

The microneedle device 2 includes a microneedle array 21, capsule-disrupting micro-projections 22, and drug delivery channels 23. The microneedle array 21 is formed protrudingly downwardly from the bottom surface of a substrate 24 for piercing the skin of a subject. The capsule-disrupting micro-projections 22 are formed upwardly from the top surface of the substrate, i.e., a surface opposite to the bottom surface on which the microneedle array 21 is formed. The drug delivery channels 23 are formed therein so as to allow an active agent to be delivered from the top surface of the substrate 24 to the bottom surface of the substrate 24 therethrough so that the active agent from the microneedles permeates the skin.

Since the drug-containing capsule 3 is positioned spaced apart from the microneedle device 2, it is not brought into contact with the capsule-disrupting micro-projections 22. As a result, the drug-containing capsule 3 can be prevented from being disrupted by the capsule-disrupting micro-projections 22, thereby eliminating the possibility of the loss of the active agent in the capsule during the storage and distribution thereof.

On the other hand, during the use of the microneedle drug delivery system, the drug-containing capsule 3 is moved to a position where it can be brought into contact with the capsule-disrupting micro-projections 22. Then, when the drug-containing capsule 3 is pressed with a certain pressure, it is disrupted or broken by the capsule-disrupting micro-projections 22 so that the drug contained in the capsule 3 is delivered into the skin through the drug delivery channels 23 by means of the microneedle array 21.

Further, the region in which the microneedle device 2 is seated defines a concave-shaped drug-accommodating portion 11 at the bottom wall of the housing 1 so that the drug flowing out of the drug-containing capsule 3 being disrupted is not diffused to the other places but can be more effectively delivered into the body through the drug delivery channels 23.

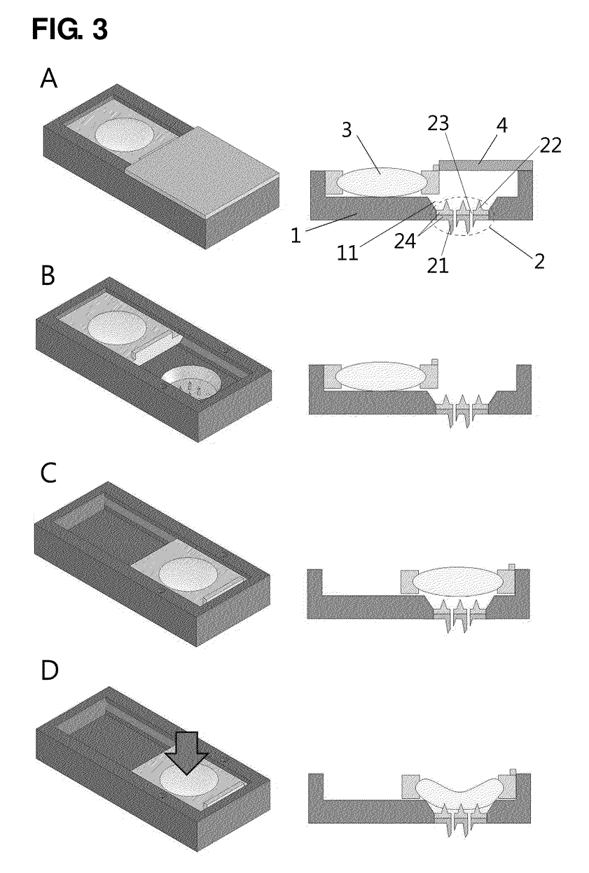

FIG. 3A to 3D are perspective and cross-sectional views illustrating the use example of a microneedle drug delivery system according to an embodiment of the present invention in which the housing 1 further includes a fixing cap 4 disposed on the top thereof so as to prevent the drug-containing capsule 3 from being moved to the microneedle device 2 therein. In this microneedle drug delivery system, the drug-containing capsule 3 is fixed in a position of being spaced apart from the microneedle device 2 during the storage or distribution as shown in FIG. 3(A).

For the purpose of the use of the microneedle drug delivery system, first, the fixing cap 4 is removed from the housing as shown in FIG. 3(B), and then the drug-containing capsule 3 is moved to a position where the microneedle device 2 is mounted in the housing 1 as shown in FIG. 3(C). In this state, when a certain pressure is exerted on the top of the drug-containing capsule 3, the capsule-disrupting micro-projections 22 disrupt or pierce the bottom of the drug-containing capsule 3 to cause the drug (or active agent) to flow out of the capsule and simultaneously the microneedle array 21 penetrates the skin as shown in FIG. 3(D). The drug flows downwardly from the capsule 3 toward the skin through the drug delivery channels, and then penetrates through the stratum corneum skin layer for in vivo delivery with an aid of the microneedle array 21.

Although not shown separately in FIG. 3, a cover integral with or separate from the fixing cap 4 may be disposed on the top of the housing 1 in such a fashion as to be positioned above the drug-containing capsule 3 so as to prevent the drug-containing capsule from escaping from the housing 1 and further protect the drug-containing capsule 3 from an external impact.

In addition, although not shown in FIG. 3, a microneedle tip protection cover such as a film or lid may be provided at the bottom of the microneedle array so as to protect the microneedle array 21 of the microneedle device 2 protruded outwardly from the housing 1 so that the microneedle tip protection cover can be removed from the microneedle array in use.

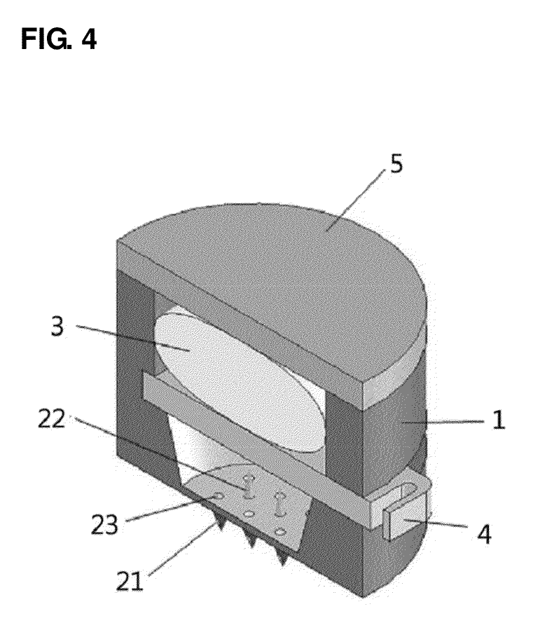

FIG. 4 is a perspective cross-sectional view illustrating a microneedle drug delivery system according to another embodiment of the present invention. In FIG. 4, similar to the case of FIG. 3, the drug-containing capsule 3 is positioned spaced apart from the capsule-disrupting micro-projections 22 of the microneedle device 2. However, the drug-containing capsule 3 is horizontally spaced apart from the capsule-disrupting micro-projections 22 in the system of FIG. 3, but is vertically spaced apart from the capsule-disrupting micro-projections 22 in the system of FIG. 4. Likewise, a fixing cap 4 is positioned to prevent the contact between the drug-containing capsule 3 and the capsule-disrupting micro-projections 22. During the use of the microneedle drug delivery system, the fixing cap 4 is removed and then a certain pressure is applied to the top of the drug-containing capsule 3 so that the drug can be delivered in vivo through the microneedles. In addition, as shown in FIG. 4, a cover 5 is disposed on the top of the housing 1 so as to prevent the drug-containing capsule 3 from escaping from the housing 1 and protect the drug-containing capsule 3 from an external impact.

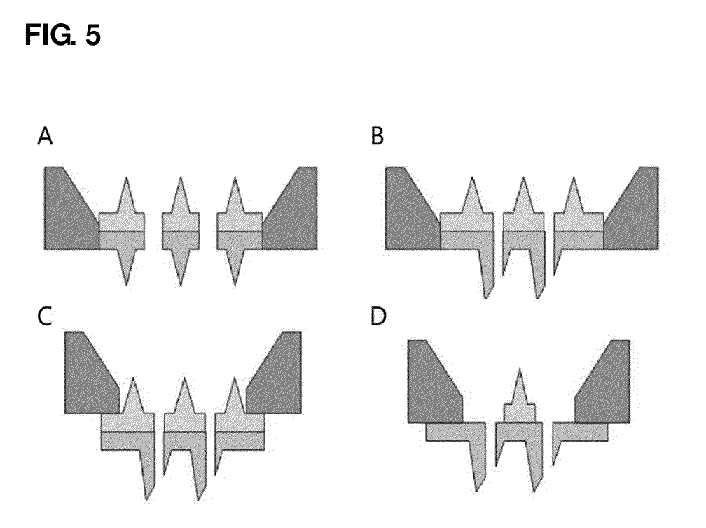

FIG. 5A to 5D are cross-sectional views illustrating examples of a microneedle device used in a microneedle drug delivery system according to an embodiment of the present invention.

A connection portion between the microneedle device 2 and the bottom wall of the housing 1 in which the microneedle device 2 is mounted is preferably inclined downwardly as shown in FIG. 5 to prevent the drug flowing out of the capsule 3 from leaking to other places, so that it forms the shape of a concaved recess with the microneedle device 2.

As described above, the microneedle device 2 includes the microneedle array 21 formed protrudingly downwardly from the bottom wall of the housing 1 and the capsule-disrupting micro-projections 22 formed upwardly from the bottom wall of the housing. Moreover, the housing 1 includes a plurality of drug delivery channels 23 formed in the bottom wall thereof so that the drug flowing out of the drug-containing capsule 3 can be delivered from the housing 1 to the outside through the microneedle device 2.

The drug delivery channels 23 may be penetratingly formed in the microneedles as shown in FIGS. 5(B), 5(C) and 5(D), and may be formed in the substrate, but not in the microneedles as shown in FIG. 5 (A). In the case where the drug delivery channels are formed in the microneedles, the drug can be directly introduced into the body, thereby leading to an increase in the drug delivery efficiency, but a high-level technology is needed to manufacture the microneedles, thereby disadvantageously leading to an increase in the manufacturing cost. In the meantime, the formation of the drug delivery channels in the substrate is performed relatively easily but is more advantageous than the formation of the drug delivery channels in the microneedles in view of the drug delivery efficiency. Thus, the drug delivery channels are preferably formed in the substrate at an upper portion of the microneedle device. In the case where the drug delivery efficiency or the drug delivery amount is not significantly important, however, the drug delivery channels can be formed penetratingly in the substrate as shown in FIG. 5(A), and in the case where the drug delivery efficiency or the drug delivery amount is important despite an increase in the manufacturing cost, the drug delivery channels can be formed penetratingly in the microneedles as shown in FIGS. 5(B) to 5(D) at an upper portion of the microneedle device.

Meanwhile, the housing 1 and the microneedle device 2 may be integrally formed with each other. However, since the manufacture of the microneedle array 21 of the microneedle device 2 and the capsule-disrupting micro-projections 22 require a precision machining technique or a precision molding technique, the respective elements of the microneedle array 21 and the micro-projections 22 may be manufactured separately and then be assembled together as shown in FIGS. 5(C) and 5(D) so as to facilitate the manufacture of the microneedle array 21 and the micro-projections 22.

According to the present invention, it is possible to provide a microneedle drug delivery system which can be carried and used more simply and conveniently, and can effectively deliver an active agent (or drug) into a subject in need.

While the present invention has been described with reference to the particular illustrative embodiments, it is not to be restricted by the embodiments but only by the appended claims. It is to be appreciated that those skilled in the art can change or modify the embodiments without departing from the scope and spirit of the present invention.

1. A microneedle drug delivery system, comprising:
a housing having an opening formed in the bottom wall thereof;
a microneedle device seated in the opening of the housing in such a fashion as to be hermetically sealed with the bottom wall of the housing, the microneedle device including a substrate, a microneedle array formed protrudingly downwardly from the bottom surface of the substrate, one or more capsule-disrupting micro-projections formed upwardly from the top surface of the substrate, and one or more drug delivery channels formed therein so as to allow a drug to be delivered from the top surface of the substrate to the bottom surface of the substrate therethrough; and
a drug-containing capsule mounted in the housing in such a fashion as to be positioned spaced apart from the microneedle device, and adapted to be moved to a position where the drug-containing capsule can come into contact with the one or more capsule-disrupting micro-projections to allow the drug-containing capsule to be disrupted by the one or more capsule-disrupting micro-projections.

2. The microneedle drug delivery system according to claim 1, wherein the region in which the microneedle device is seated defines a concave-shaped drug-accommodating portion at the bottom wall of the housing.

3. The microneedle drug delivery system according to claim 1, wherein the drug-containing capsule is movable in a horizontal direction in the housing.

4. The microneedle drug delivery system according to claim 1, wherein the drug-containing capsule is movable in a vertical direction in the housing.

5. The microneedle drug delivery system according to claim 1, wherein the drug delivery channels are formed penetratingly in the substrate and the microneedles.

6. The microneedle drug delivery system according to claim 1, wherein the drug delivery channels are formed penetratingly in the substrate.

7. The microneedle drug delivery system according to claim 1, wherein the housing further comprises a fixing cap adapted to be removable therefrom and disposed on the top thereof so as to prevent displacement of the drug-containing capsule therein.

8. The microneedle drug delivery system according to claim 1, wherein the housing further comprises a cover disposed on the top thereof so as to protect the top of the drug-containing capsule.

9. The microneedle drug delivery system according to claim 1, wherein the microneedle array further comprises a microneedle tip protection cover provided at the bottom thereof.

10. The microneedle drug delivery system according to claim 1, wherein the drug-containing capsule is divided into two internal compartments.

11. The microneedle drug delivery according to claim 1, wherein each of the capsule-disrupting micro-projections comprises a solid or gel-type drug layer formed thereon, and the drug-containing capsule is filled with a solvent for dissolving the drug.

12. The microneedle drug delivery system according to claim 2, wherein the drug-containing capsule is movable in a horizontal direction in the housing.

13. The microneedle drug delivery system according to claim 2, wherein the drug-containing capsule is movable in a vertical direction in the housing.

14. The microneedle drug delivery system according to claim 2, wherein the drug delivery channels are formed penetratingly in the substrate and the microneedles.

15. The microneedle drug delivery system according to claim 2, wherein the drug delivery channels are formed penetratingly in the substrate.

16. The microneedle drug delivery system according to claim 2, wherein the housing further comprises a fixing cap adapted to be removable therefrom and disposed on the top thereof so as to prevent displacement of the drug-containing capsule therein.

17. The microneedle drug delivery system according to claim 2, wherein the housing further comprises a cover disposed on the top thereof so as to protect the top of the drug-containing capsule.

18. The microneedle drug delivery system according to claim 2, wherein the microneedle array further comprises a microneedle tip protection cover provided at the bottom thereof.

19. The microneedle drug delivery system according to claim 2, wherein the drug-containing capsule is divided into two internal compartments.

20. The microneedle drug delivery according to claim 2, wherein each of the capsule-disrupting micro-projections comprises a solid or gel-type drug layer formed thereon, and the drug-containing capsule is filled with a solvent for dissolving the drug.
